(19) United States
(12) Patent Application Publication
(10) Pub. No.: US 2021/0106376 A1
(43) Pub. Date: Apr. 15, 2021

(54) SURGICAL CUTTING INSTRUMENT WITH GUARD

(71) Applicant: Covidien LP, Mansfield, MA (US)

(72) Inventors: Nikolai D. Begg, Wellesley, MA (US); Ronald L. Green, Bethel, CT (US)

(21) Appl. No.: 17/067,485

(22) Filed: Oct. 9, 2020

Related U.S. Application Data
(60) Provisional application No. 62/913,399, filed on Oct. 10, 2019.

(51) Int. Cl. A61B 18/14

(52) U.S. Cl. CPC A61B 18/1442; A61B 2018/00196; A61B 2018/1455

(57) ABSTRACT

A surgical instrument includes a housing and a motor. An elongated member extends from the housing. An end effector is at a distal end of the elongated member. The end effector includes a jaw member including a guard. The guard has a number of serrations extending from a proximal end of the jaw member to a distal end of the jaw member. The guard defines corresponding pockets between the serrations. The guard is coupled to the motor to induce reciprocation of the guard relative to the blade upon activation of the motor. A blade is recessed within the guard to expose a cutting edge of the blade between the pockets. The pockets engage tissue. A treatment tip is at a distal end of the guard. The treatment tip connects to an energy source. The treatment tip treats tissue upon activation of the treatment tip.

### CROSS-REFERENCE TO RELATED APPLICATIONS

The present application claims priority to and the benefit of U.S. Provisional Application No. 62/913,399 filed Oct. 10, 2019. The entire contents of which are incorporated by reference herein.

### BACKGROUND

### Technical Field

The present disclosure relates generally to surgical apparatuses for use in minimally invasive surgical procedures, such as endoscopic and/or laparoscopic procedures, or open procedures, and more particularly, the present disclosure relates to a surgical cutting instrument having a guard.

### Discussion of Related Art

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy and laryngobronchoscopy, just to name a few. In these procedures, trocars are utilized for creating incisions through which the endoscopic surgery is performed. Trocar tubes or cannula devices are extended into and left in place in the abdominal wall to provide access for endoscopic surgical tools. A camera or endoscope is inserted through a trocar tube to permit the visual inspection and magnification of the body cavity. The surgeon can then perform diagnostic and/or therapeutic procedures at the surgical site with the aid of specialized instrumentation, such as forceps, graspers, cutters, applicators, and the like, which are designed to fit through additional cannulas.

Minimally-invasive or open surgical procedures may each be used for partial or total retrieval of a tissue specimen from an internal body cavity, or for careful dissection of a particular tissue or area without dissecting adjacent organs or vessels. For example, dense tissue adhesions may be removed through sharp dissection techniques including a scalpel, scissors or other surgical cutting devices. Preferential dissection of “tissue strings” associated with dense tissue adhesions may be performed by selectively cutting particular tissue regions.

### SUMMARY

In accordance with an aspect of the present disclosure, a surgical instrument includes a housing and a motor within the housing. An elongated member extends from a distal end of the housing. An end effector is at a distal end of the elongated member. The end effector includes a jaw member including a guard. The guard has a number of serrations extending from a proximal end of the jaw member to a distal end of the jaw member. The guard defines corresponding pockets between the serrations. The guard is coupled to the motor to induce reciprocation of the guard relative to the blade upon activation of the motor. A blade is recessed within the guard to expose a cutting edge of the blade between the pockets. The pockets engage tissue. A treatment tip is at a distal end of the guard. The treatment tip connects to an energy source. The treatment tip treats tissue upon activation of the treatment tip.

In some aspects, the treatment tip may be electrically conductive, resistive or ultrasonic.

In some aspects, the serrations of the guard include a geometry configured to direct tissue into the corresponding pockets when the guard is moved across tissue to induce cutting by the blade.

In some aspects, the geometry of each serration includes angled surfaces to direct tissue into the corresponding pockets when the guard is moved across tissue to induce cutting by the blade. The angled surfaces may include proximal-facing surfaces or distal-facing surfaces.

In some aspects, the cutting edge is sharpened to induce mechanical cutting.

In some aspects, the cutting edge is substantially dull to limit mechanical cutting and induce electrical cutting.

In some aspects, the blade is adapted to connect to a first energy source or a second energy source to induce cutting. The blade may cut tissue via electrical cutting, ultrasonic cutting, microwave cutting, optical cutting, or resistive cutting.

In accordance with an aspect of the present disclosure, the jaw member includes the guard including the serrations extending from the proximal end of the jaw member to the distal end of the jaw member. The guard defines a corresponding plurality of pockets between the serrations. The blade is positioned within the guard. The blade is coupled to the motor to induce reciprocation of the blade relative to the guard upon activation of the motor. The blade is recessed within the guard to expose the cutting edge of the blade between the pockets. The pockets are arranged to engage tissue

In some aspects, the serrations of the guard include a geometry to direct tissue into the corresponding pockets when the blade is moved across tissue to induce cutting by the blade. For example, the geometry of each serration includes angled surfaces to direct tissue into the corresponding pockets when the blade is moved across tissue to induce cutting by the blade. The angled surfaces may include distal-facing surfaces or proximal facing surfaces.

### BRIEF DESCRIPTION OF THE DRAWINGS

The accompanying drawings, which are incorporated in and constitute a part of this specification, illustrate embodiments of the present disclosure and, together with the detailed description below, serve to further explain the present disclosure, in which:

FIG. 1 is a front, perspective view of a surgical cutting instrument configured for use in accordance with the present disclosure;

FIG. 2 is a front, perspective view of a first end effector employable by the surgical cutting instrument of FIG. 1 in accordance with the present disclosure; and

FIG. 3 is a front, perspective view of a second end effector employable by the surgical cutting instrument of FIG. 1 in accordance with the present disclosure.

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

“About” or “approximately” or “substantially” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±30%, 20%, 10%, 5% of the stated value.

Descriptions of technical features or aspects of an exemplary embodiment of the present disclosure should typically be considered as available and applicable to other similar features or aspects in another exemplary embodiment of the present disclosure. Accordingly, technical features described herein according to one exemplary embodiment of the present disclosure may be applicable to other exemplary embodiments of the present disclosure, and thus duplicative descriptions may be omitted herein.

Exemplary embodiments of the present disclosure will be described more fully below (e.g., with reference to the accompanying drawings). Like reference numerals may refer to like elements throughout the specification and drawings. The surgical cutting instrument described herein may be particularly useful in minimally invasive surgical procedures, such as endoscopic and/or laparoscopic procedures, or open procedures.

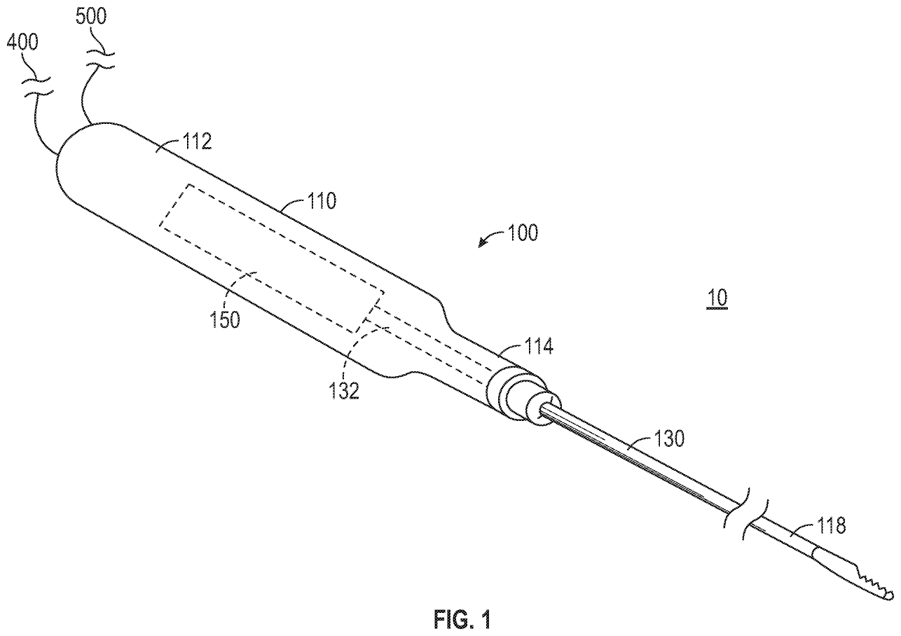

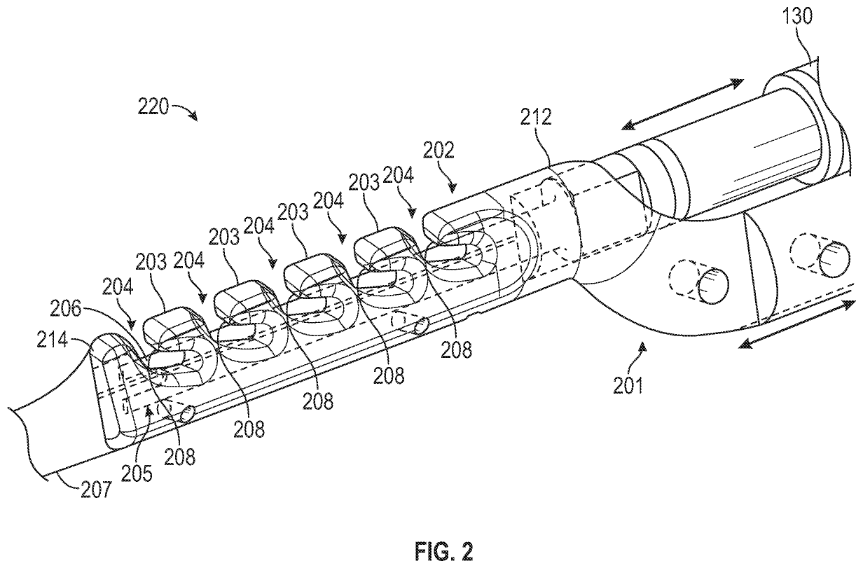

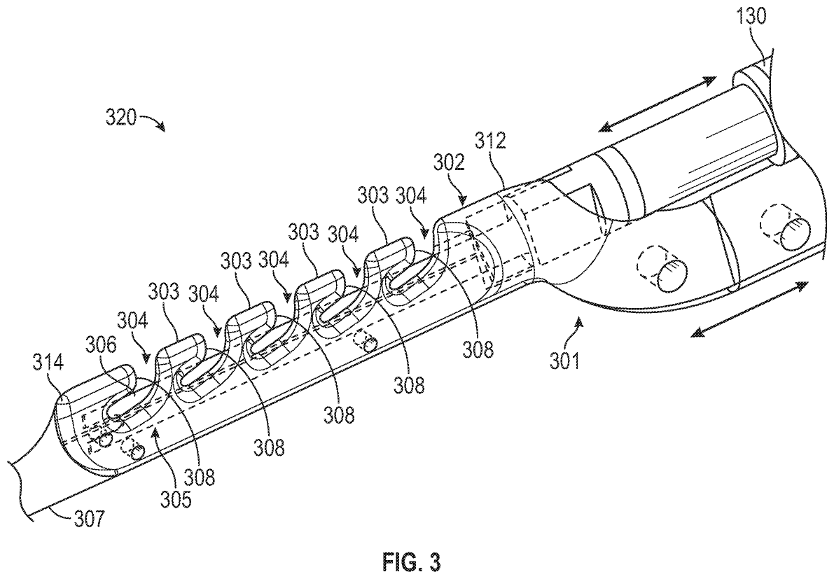

Referring to FIGS. 1 to 3, a surgical instrument 10 includes a housing 110 and a motor 150 within the housing 110. An elongated member 130 extends from a distal end 114 of the housing 110. An end effector (e.g., end effector 220 or 320 illustrated in FIG. 2 or 3, respectively) is disposed at a distal end 118 of the elongated member 130. In some embodiments, the elongated member 130 may include a portion 132 extending at least partially through the housing 110 to connect with motor 150. Thus, an actuating motion of the motor 150 may be translated through the housing 110 and into a guard 202 or a blade 205 disposed within the end effector (e.g., end effector 220 or 320 illustrated in FIG. 2 or 3, respectively) to create a reciprocating motion in the guard 202 or the blade 205, as described in more detail below. The phrases “surgical instrument” and “surgical cutting instrument” may be used interchangeably herein.

Referring to FIGS. 1 and 2, the end effector 220 includes a jaw member 201 including a guard 202. The guard 202 includes a series of serrations 203 extending from a proximal end 212 of the jaw member 201 to a distal end 214 of the jaw member 201. The guard 202 defines corresponding pockets 204 between adjoining serrations 203. The guard 202 is coupled to the motor 150 to induce reciprocation of the guard 202 relative to the blade 205 upon activation of the motor 150. The blade 205 is recessed within the guard 202 to expose a cutting edge 206 of the blade 205 within each pocket 204. The serrations 203 are configured to engage tissue and direct the tissue into the corresponding pockets 204 and into contact with the blade 205 to facilitate cutting of the tissue. A treatment tip 207 extends distally from a distal end 214 of the guard 202. The treatment tip 207 may be connected to an energy source (e.g., energy source 400) or may be coupled to an internal energy source (e.g., battery). The treatment tip 207 is configured to treat tissue (e.g., by coagulation, ultrasonic, resistive heating, etc.) upon activation thereof. The treatment tip 207 may be, for example, an electrically conductive, resistive or ultrasonic tip.

In accordance with another embodiment of the present disclosure, the blade 205 may be coupled to the motor 150 to induce reciprocation of the blade 205 relative to the guard 202 upon activation of the motor 150. The serrations 203 of the guard 202 include a geometry to direct tissue into the corresponding pockets 204 and across the blade 205 induce cutting by the blade 205. Alternatively, tissue may be directed into the corresponding pockets 204 when the guard 202 is moved relative to the blade 205 to induce cutting.

In embodiments, the geometry of each serration 203 includes angled surfaces 208 configured to direct tissue into the corresponding pockets 204. The angled surfaces 208 may increase pressure between the cutting edge 206 of the blade 205 and tissue being cut by pinching the tissue between the angled surfaces 208 and the cutting edge 206. The angled surfaces 208 in end effector 220 are distal-facing surfaces. Thus, the end effector 220 may be particularly useful for cutting tissue by advancing the surgical cutting instrument 10 along a distal direction as tissue is directed into the cutting edge 206 of the blade 205 by the distal-facing surfaces of the guard 202.

An end effector 320 is described below with reference to FIGS. 1 and 3. The end effector 320 is substantially the same as the end effector 220 unless otherwise indicated (e.g., end effector 320 includes proximal-facing angled surfaces 308). Thus technical features described with respect to end effector 220 are similarly available to end effector 320 wherever technically feasible.

Referring to FIGS. 1 and 3, the end effector 320 includes a jaw member 301 including a guard 302. The guard 302 includes a series of serrations 303 extending from a proximal end 312 of the jaw member 301 to a distal end 314 of the jaw member 301. The guard 302 defines corresponding pockets 304 between adjoining serrations 303.

The guard 302 is coupled to the motor 150 to induce reciprocation of the guard 302 relative to the blade 305 upon activation of the motor 150. The blade 305 is recessed within the guard 302 to expose a cutting edge 306 of the blade 305 within each pocket 304. The serrations 303 engage tissue and direct the tissue into the pockets 304 to contact the blade 305 to facilitate cutting of the tissue. A treatment tip 307 extends from a distal end 314 of the guard 302. The treatment tip 307 may be connected to an energy source (e.g., energy source 400) or may be coupled to an internal energy source (e.g., battery). The treatment tip 307 is configured to treat tissue (e.g., by coagulation, ultrasonic, resistive heating, etc.) upon activation thereof.

In accordance with another embodiment of the present disclosure, the blade 305 may be coupled to the motor 150 to induce reciprocation of the blade 305 relative to the guard 302 upon activation of the motor 150. The serrations 303 of the guard 302 include a geometry to direct tissue into the corresponding pockets 304 and across the blade 305 to induce cutting by the blade 305. Alternatively, tissue may be directed into the corresponding pockets 304 when the guard 302 is moved relative to the blade 305 to induce cutting.

The geometry of each serration 303 may be configured to include angled surfaces 308 to direct tissue into the corresponding pockets 304. The angled surfaces 308 may increase pressure between the cutting edge 306 of the blade 305 and tissue being cut by pinching the tissue between the angled surfaces 308 and the cutting edge 306. The angled surfaces 308 of end effector 320 are proximal-facing surfaces. Thus, the end effector 320 may be particularly useful for cutting tissue by advancing the surgical cutting instrument 10 along a proximal direction as tissue is directed into the cutting edge 306 of the blade 305 by the proximal-facing surfaces of the guard 302.

Referring to FIGS. 1-3, in some embodiments, the treatment tip (e.g., tip 207 or 307) is electrically connected to a switch 50 operably disposed on the housing 110. The switch 50 is activatable to supply electrosurgical energy to the treatment tip 207, 307 using an energy algorithm. The energy algorithm includes a cutting algorithm, a coagulating algorithm and/or a blending algorithm. Thus, the treatment tip 207, 307 may be used to “spot treat” a desired area without the need to employ another instrument.

The cutting edge (e.g., cutting edge 206 or 306) may be sharpened to facilitate mechanical cutting. The cutting edge (e.g., cutting edge 206 or 306) may be substantially dull to limit mechanical cutting and induce electrical cutting. In this instance, the blade 205, 305 would be coupled to an electrosurgical energy source. The blade (e.g., blade 205 or 305) may be configured to cut tissue via electrical cutting, ultrasonic cutting, microwave cutting, optical cutting, or resistive cutting.

The blade (e.g., blade 205 or 305) may be adapted to connect to a first energy source 400 (e.g., a generator) or a second energy source 500 (e.g., a generator) to selectively induce cutting. The first energy source 400 may be the same energy source as the energy source connected with the treatment tip (e.g., tip 207 or 307). The second energy source 500 may be a separate energy source from the first energy source 400. The second energy source 500 may supply energy to the blade, while the first energy source 400 supplies energy to the treatment tip 207, 307. A single energy source (e.g., energy source 400 or 500) may selectively apply energy to the blade (e.g., blade 205 or 305) and/or the treatment tip 207 or 307. Independent switches operably disposed on the housing 110 may independently control a supply of energy to the blade 205, 305 or the treatment tip 207, 307, respectively.

Referring particularly to FIGS. 1 and 2, the distal-facing surface 208 of guard 202 may be particularly useful for cutting tissue by advancing the surgical cutting instrument 10 in a distal direction. By advancing the surgical cutting instrument 10 in a distal direction, tissue is forced into the pockets 204 along the distal-facing angled surfaces 208 and into contact with the cutting edge 206 of blade 205. This may occur while the blade 205 and/or the guard 202 move in a longitudinal reciprocating fashion relative to the end effector 220 (see, e.g., the bidirectional arrows illustrated in FIGS. 2 and 3).

Increased pressure between cutting tissue and the cutting edge 206 is generated at a point along the cutting edge 206 in substantially immediate proximity to a lower end of the distal-facing angled surfaces 208 (e.g., at a point where the angled surfaces 208 cross the cutting edge 206). As a result thereof, tissue may be cut along a desired plane without the blade 205 contacting adjacent organs or vessels. For example, “strings” or particular regions of tissue adhesions may be precisely cut by use of a particular pocket 204 of end effector 220 directing tissue into precise contact with the cutting edge 206 of blade 205.

Referring particularly to FIGS. 1 and 3, end effector 320 may be employed in substantially the same fashion as end effector 220, except that end effector 320 is particularly useful for cutting tissue in a proximal direction.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely controls the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical instrument, comprising:
a housing;
a motor operably disposed within the housing;
an elongated member extending from a distal end of the housing; and
an end effector operably disposed at a distal end of the elongated member, the end effector including:
a jaw member including a guard having a plurality of serrations extending from a proximal end of the jaw member to a distal end of the jaw member, the guard defining a corresponding plurality of pockets between the serrations, the guard operably coupled to the motor to induce reciprocation thereof relative to the blade upon activation of the motor;
a blade operably disposed within the guard, the blade recessed within the guard to expose a cutting edge of the blade between the plurality of pockets configured to engage tissue; and
a treatment tip disposed at a distal end of the guard, the treatment tip adapted to connect to an energy source and configured to treat tissue upon activation thereof.

2. The surgical instrument of claim 1, wherein the plurality of serrations of the guard includes a geometry configured to direct tissue into the corresponding plurality of pockets when the guard is moved across tissue to induce cutting by the blade.

3. The surgical instrument of claim 1, wherein the geometry of each serration includes angled surfaces to direct tissue into the corresponding plurality of pockets when the guard is moved across tissue to induce cutting by the blade.

4. The surgical instrument of claim 3, wherein the angled surfaces include proximal-facing surfaces.

5. The surgical instrument of claim 3, wherein the angled surfaces include distal-facing surfaces.

6. The surgical instrument of claim 1, wherein the cutting edge is sharpened to induce mechanical cutting.

7. The surgical instrument of claim 1, wherein the cutting edge is substantially dull to limit mechanical cutting and induce electrical cutting.

8. The surgical instrument of claim 1, wherein the blade is adapted to connect to the energy source or a second energy source to induce cutting.

9. The surgical instrument of claim 8, wherein the blade cuts tissue via electrical cutting, ultrasonic cutting, microwave cutting, optical cutting, or resistive cutting.

10. The surgical instrument of claim 1, wherein the treatment tip is electrically conductive, resistive or ultrasonic.

11. A surgical instrument, comprising:
a housing;
a motor operably disposed within the housing;
an elongated member extending from a distal end of the housing; and
an end effector operably disposed at a distal end of the elongated member, the end effector including:
a jaw member including a guard having a plurality of serrations extending from a proximal end of the jaw member to a distal end of the jaw member, the guard defining a corresponding plurality of pockets between the serrations;
a blade operably disposed within the guard and operably coupled to the motor to induce reciprocation thereof relative to the guard upon activation of the motor, the blade recessed within the guard to expose a cutting edge of the blade between the plurality of pockets configured to engage tissue; and
an treatment tip disposed at a distal end of the guard, the treatment tip adapted to connect to an energy source and configured to treat tissue upon activation thereof.

12. The surgical instrument of claim 11, wherein the plurality of serrations of the guard includes a geometry configured to direct tissue into the corresponding plurality of pockets when the blade is moved across tissue to induce cutting by the blade.

13. The surgical instrument of claim 11, wherein the geometry of each serration includes angled surfaces to direct tissue into the corresponding plurality of pockets when the blade is moved across tissue to induce cutting by the blade.

14. The surgical instrument of claim 13, wherein the angled surfaces include proximal-facing surfaces.

15. The surgical instrument of claim 13, wherein the angled surfaces include distal-facing surfaces.

16. The surgical instrument of claim 11, wherein the cutting edge is sharpened to induce mechanical cutting.

17. The surgical instrument of claim 11, wherein the cutting edge is substantially dull to limit mechanical cutting and induce electrical cutting.

18. The surgical instrument of claim 11, wherein the blade is adapted to connect to the energy source or a second energy source to induce cutting.

19. The surgical instrument of claim 18, wherein the blade cuts tissue via electrical cutting, ultrasonic cutting, microwave cutting, optical cutting, or resistive cutting.

20. The surgical instrument of claim 11, wherein the treatment tip is electrically conductive, resistive or ultrasonic.
